(19) United States
(12) Patent Application Publication
(10) Pub. No.: US 2024/0044926 A1
(43) Pub. Date: Feb. 8, 2024

(54) SYSTEM AND METHOD FOR CONDUCTING AUTOMATED CLINICAL DIAGNOSTIC CROSSOVER STUDIES

(71) Applicant: Bio-Rad Laboratories, Inc., Hercules, CA (US)

(72) Inventors: John Yundt-Pacheco, Fairview, TX (US); Curtis Parvin, Garland, TX (US); Nico VandePoele, Aliso Viejo, CA (US)

(73) Assignee: Bio-Rad Laboratories, Inc., Hercules, CA (US)

(21) Appl. No.: 18/258,638

(22) PCT Filed: Dec. 21, 2020
(86) PCT No.: PCT/US2020/066424

§ 371 Date: Jun. 21, 2023

(51) Int. Cl. G01N 35/00

(52) U.S. Cl. CPC G01N 35/00613; G01N 35/00871; G01N 2035/0091; G01N 2035/00881

(57) ABSTRACT

A clinical diagnostic analyzer for performing an automated crossover study on quality control (QC) material includes a processor, memory, measurement hardware, and an input panel/display. The analyzer prompts a user to load a QC specimen, and to instigate testing and analysis to determine a mean and a standard deviation for the new material. Associated methods for using one or more clinical diagnostic analyzers to calculate a new mean and standard deviation for a new QC material, reduce error in the calculated mean value, and to reduce the total number of days to complete a crossover study are also disclosed.

### CROSS-REFERENCE TO RELATED APPLICATIONS

This application is a 371 national phase of, and claims the benefit of, International Application Number PCT/US2020/066424, filed Dec. 21, 2020, the disclosure of which is hereby incorporated herein in its entirety by reference.

### BACKGROUND OF THE INVENTION

The present invention relates to generally to clinical diagnostic analyzers, and more particularly to systems and methods for conducting automated cross over studies in such analyzers.

Clinical diagnostic laboratories use various quality control schemes to ensure that the clinical diagnostic processes employed and the clinical diagnostic analyzers used to analyze patient specimens, or other test specimens, provide accurate diagnostic results. One common quality control scheme involves testing quality control (QC) materials having known properties using the same analyzers and processes that are used to test patient specimens. Running such quality control tests with material having known properties ensures that the clinical diagnostic analyzers used to perform the test provide expected and accurate results, or provide results within a predetermined range or specification, and likewise ensures that the reagents and processes used in conjunction with the analyzers provide expected results.

While quality control testing using control material having known properties is generally useful, statistical control issues arise when the control materials must be replenished. Because the control materials have a limited lifetime, and because QC testing using the control material consumes that material, laboratories must regularly deal with obtaining and using new lots of control materials, requiring them to crossover and begin using the new lot of QC material. Crossing over to the new QC material is a substantial undertaking for a laboratory, as the reliability and accuracy of the new control material must be ensured before proceeding with further testing relying on the new control materials. Even though the new lot of QC control material will have similar properties to the previous lot, the variations between lots will affect the accuracy of the testing, particularly until a sufficient number of tests can be run on the new QC materials. Thus, laboratories must engage in crossover studies to verify the accuracy of the new materials before the desired accuracy of the testing can be ensured. Such crossover studies must be done for any change in control materials, because even with control materials that have insert ranges, i.e., assayed control materials, insert ranges are only intended for laboratories to quickly determine if they are in control, they are not intended for use for performance monitoring.

Crossover studies involve determining the statistical behavior of a new lot of QC control material, namely, estimating the mean and standard deviation (SD) of the new material. In order to obtain that mean and SD measurement, the general approach for crossover studies has been to evaluate samples and collect data of the new control material over time until sufficient data has been collected to compute the mean and SD from the collected data, then, once calculated, using and assigning that calculated mean and SD for future control testing using the new quality control materials.

One generally accepted method of making that initial assessment is that described in Statistical Quality Control for Quantitative Measurement Procedures: Principles and Definitions; Approved Guideline—Third Edition, which calls for making a minimum of at least twenty different measurements of control material, for each control level, on separate days. That generally accepted method thus requires collecting at least twenty data points per control level, over a period of twenty days. Thus, for example, for a trilevel control involving thirty separate analytes, ninety separate studies must be conducted with a data point collected for each individual test. That collected data is then used to estimate a mean and SD for the new lot of material. In addition to the time required, such studies incur considerable expense for the laboratory, with each molecular data point collected incurring costs of $200 or more. Such studies are also labor intensive. As there is no standardized system for conducting such crossover studies, most laboratories typically manually process the collected data using a spreadsheet, and manually input the data to calculate the mean and SD of the new control material.

Even after incurring the time, expense, and inefficiency of conducting crossover studies in accordance with the generally recommended procedures, the results of those studies do not have the accuracy desired or required by the laboratory. For example, while twenty data points is sufficient to determine the mean of the new material, collecting that number of data points is not necessary and is thus inefficient, as the mean can be determined by using just ten data points. Thus, the generally recommended crossover study method incurs unnecessary testing and expense with respect to determining the mean. Furthermore, twenty data points is insufficient to determine the SD with a desired level of accuracy, typically eighty data points are required. Using the generally recommended method thus typically results in estimated SD's having a high error margin.

Recognizing the above limitations, the industry has suggested an alternative for determining the SD of the new control material based on using only ten data points by incorporating the mean and SD of the old material, using the equation SD_{new}=(MEAN_{new}*CV_{old})/100, where CV_{old}=SD_{old}/MEAN_{old}. However, while that that alternative determination requires a fewer number of data points, and thus takes less time, the results using that method still incur potential inaccuracies in the mean calculation (see, e.g., C24 Statistical Quality Control for Quantitative Measurement Procedures: Principles and Definitions, 4^{th} Edition).

Thus, it is apparent that current methods of conducting crossover studies are insufficient and that there remains a need in the art for an improved system and method for conducting crossover studies that increases the accuracy and reduces the time and expense incurred as compared to generally known methods.

### BRIEF SUMMARY OF THE INVENTION

The present invention is directed to a system and method for conducting automated crossover studies in clinical diagnostic analyzers. In an exemplary embodiment, the system and method of the present invention employs one or more clinical diagnostic analyzers to test new quality control material and calculate new mean and standard deviation values for the new QC material.

In one aspect, a clinical diagnostic analyzer for performing an automated crossover study includes a processor, memory, measurement hardware, and an input panel/display. The analyzer prompts a user to load a QC specimen, and to instigate testing and analysis to determine a mean and a standard deviation for the new material.

In another aspect, an automated method for calculating a new mean and standard deviation for a new QC material involves collecting ten data points from the new material over a period of time and calculating the new mean and standard deviation based on the old mean and standard deviation and the newly collected data. In a further aspect, the accuracy of the calculated new mean is improved by calculating a thirty-day rolling average.

In another aspect, the total number of days required to complete a crossover study is reduced by running the same specimen on multiple clinical diagnostic analyzers so that multiple data points are collected on the same day, reducing the overall time required to collect the required number of points for the study.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings and claims. In the drawings, like reference numbers indicate identical or functionally similar elements.

### BRIEF DESCRIPTION OF THE DRAWINGS

The present invention will be described in greater detail in the following detailed description of the invention with reference to the accompanying drawings that form a part hereof, in which:

FIG. 1 depicts a block diagram of a clinical diagnostic analyzer system having a plurality of clinical diagnostic analyzers in communication with a server over a network in accordance with an exemplary embodiment of the present invention.

FIG. 2 depicts a block diagram of a single clinical diagnostic analyzer of the system of FIG. 1.

FIG. 3A is a depiction of a first exemplary prompt screen presented by the clinical diagnostic analyzer of FIG. 2.

FIG. 3B is a depiction of a second exemplary prompt screen presented by the clinical diagnostic analyzer of FIG. 2.

FIG. 3C is a depiction of a third exemplary prompt screen presented by the clinical diagnostic analyzer of FIG. 2.

FIG. 3D is a depiction of a fourth exemplary prompt screen presented by the clinical diagnostic analyzer of FIG. 2.

FIG. 4 is a flow diagram of an exemplary method for determining a mean and standard deviation of a quality control specimen in accordance with an exemplary embodiment of the present invention.

FIG. 5 is a flow diagram of an exemplary method for reducing error in the mean calculated in the method of FIG. 4.

FIG. 6 is a flow diagram of an exemplary method for conducting an automated crossover study in a reduced amount of time using a plurality of clinical diagnostic analyzers.

### DETAILED DESCRIPTION OF EXEMPLARY EMBODIMENTS

Systems and methods for conducting automated crossover studies in a clinical diagnostic analyzer in accordance with exemplary embodiments of the present invention are described herein. While the invention will be described in detail hereinbelow with reference to the depicted exemplary embodiments and alternative embodiments, it should be understood that the invention is not limited to the specific configurations shown and described in these embodiments. Rather, one skilled in the art will appreciate that a variety of configurations may be implemented in accordance with the present invention.

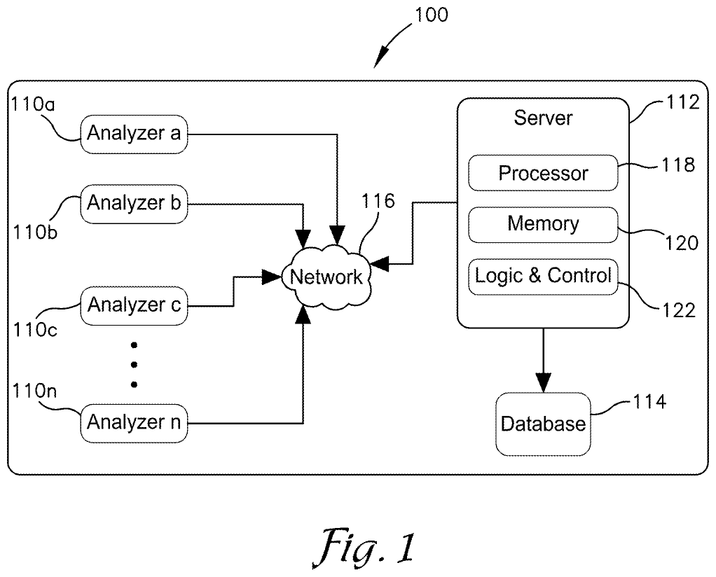

Looking first to FIG. 1, a clinical diagnostic system in accordance with an exemplary embodiment of the present invention is depicted generally by the numeral 100. The system 100 generally includes a plurality of clinical diagnostic analyzers 110a, 110b, 110c, 110n and a server 112 in communication with a database 114. The plurality of clinical diagnostic analyzers 110a, 110b, 110c, 110n are in communication with network 116, which facilitates the transmission of instructions, information, and data between each clinical diagnostic analyzer 110a, 110b, 110c, 110n and the server 112, as well as between each of the clinical diagnostic analyzers 110a, 110b, 110c, 110n and any of the other diagnostic analyzers, or between any combination of clinical diagnostic analyzers and/or the server.

Network 116 may be any local area network (LAN), wide area network (WAN), ad-hoc network, or other network configuration known in the art, or combinations thereof. For example, in the exemplary embodiment depicted in FIG. 1, network 116 may include a LAN allowing communication between the clinical diagnostic analyzers 110a, 110b, 110c, 110n, such as in a single laboratory setting having multiple clinical diagnostic analyzers, an may also include a WAN, such as the Internet or other wide area network, allowing communication between the LAN and the server 112 and/or between the clinical diagnostic analyzers and the server.

It should be understood that the configuration depicted in FIG. 1 is exemplary, and not limiting, and that the invention as described herein may be embodied in a single clinical diagnostic analyzer, in a group of clinical diagnostic analyzers co-located in a single laboratory or facility, and in group of clinical diagnostic analyzers that are geographically dispersed.

For example, multiple systems 100, each comprising one or more clinical diagnostic analyzers and servers may be located in a single laboratory, or in multiple laboratories dispersed across a facility or across the globe, all in communication via a WAN. It should be further understood that the present invention may be embodied in a single clinical diagnostic analyzer, or in a group of clinical diagnostic analyzers in communication with each other over a LAN or WAN, without a server or servers. These and other variations and embodiments will be apparent to those skilled in the art.

Server 112 preferably includes a processor 118, memory 120, and logic and control circuitry 122, all in communication with each other. Server 112 may be any server, server system, computer, or computer system known in the art, preferably configured to communicate instructions and data between the server 112 and the network, and/or to any device connected to the network, and to store and retrieve data and information to and from the database 114. Processor 118 may be any microprocessor, controller, or plurality of such devices known in the art. Processor 118 preferably runs a server operating system such as a Linux based, Windows based, or other server operating system known in the art. Preferably, the processor 118 is configured to control the operation of the server 112 in conjunction with the operating system, allowing the server to communicate with the database 114 and the network 116 and/or with devices connected to the network, such as the clinical diagnostic analyzers 110a, 110b, 110c, 110n. In some embodiments the server may control the operation of the clinical diagnostic analyzers, for example allowing operation of the analyzers during specific time periods, collecting data from the analyzers for storage in the database 114, transferring data to the analyzers for viewing and/or analysis, collecting test data from the analyzers, and providing data, instructions or prompts to the analyzers either individually or in groups.

Memory 120 may be volatile or non-volatile memory and is used to store data and information associated with the operation of the server as well as data for transmission to and from the server. For example, the memory stores the server operating system for execution by the processor 118 and may also store data associated with the clinical diagnostic analyzers 110a, 110b, 110c, 110n in communication with the server 112 over the network 116. In some embodiments the memory 120 on the server may be used as a supplement to, or in place of, the database 114.

The database 114 is preferably used to store control information relating to the operation of the server 112 and the operation and control of the clinical diagnostic analyzers 110a, 110b, 110c, 110n, and may also be used to store data relating to the processing of samples by the clinical diagnostic analyzers. For example, the database may contain instructions or programming for execution by a processor on a clinical diagnostic analyzer, or for execution on the server, or may store data related to the number of samples processed, the frequency of testing, the results of analysis performed on the analyzer, as well as data relating to the samples themselves, such as tracking information, lot numbers, sample size, sample weight, percentage of sample remaining, and the like. Preferably, the database 114 includes non-volatile storage such as hard drives, solid state memory, and combinations thereof.

Logic and control circuitry 122 provides interface circuitry to allow the processor and memory to communicate, and to provide other operational functionality to the server, such as facilitating data communications to and from the network 116.

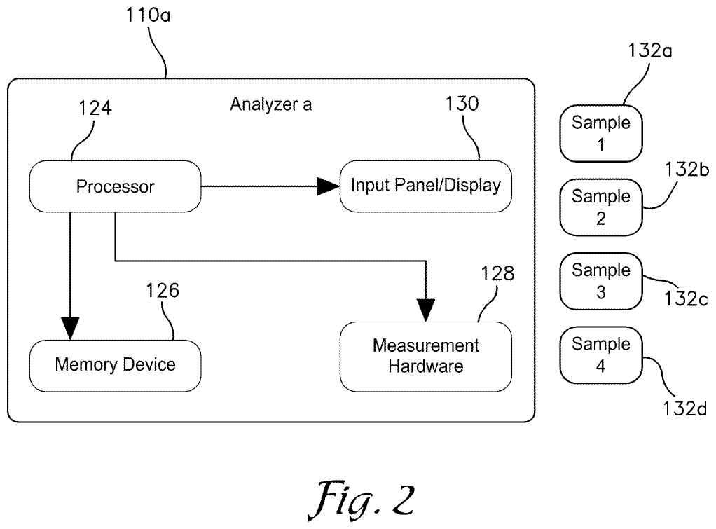

Turning to FIG. 2, a detailed view of a single clinical diagnostic analyzer 110a of the system of FIG. 1 is depicted. Clinical diagnostic analyzer 110a preferably comprises a processor 124, a memory device 126, measurement hardware 128, and an input panel/display 130.

The processor 124 may be any controller, microcontroller, or microprocessor as known in the art, and is in communication with memory device 126 which stores instructions for execution by the processor to control and communicate with the measurement hardware 128 and the input panel/display 130 to cause the clinical diagnostic analyzer to perform desired steps, such as sampling as commanding the measurement hardware to load test specimens or to perform a test on a loaded sample, or instructing or prompting an user to perform specific operations such as replacing a test sample, beginning a test, or viewing collected data. The processor 124 may also execute instructions to receive data from the measurement hardware 128 and to perform one or more analyses on the received data, and to display test results or other information on the input panel/display panel 130.

Measurement hardware 128 preferably includes a sample receptacle configured to receive one or more samples into the analyzer for testing. Preferably, the measurement hardware is configured to receive samples or specimens stored within vials, and most preferably is configured to receive a plurality of vials and to extract samples, i.e., analytes, from any desired specimen vial for testing and analysis. In further embodiments, the measurement hardware 128 may include external turntables, loaders, or other mechanisms to facilitate the loading and unloading of samples to allow samples to be loaded under command of the analyzer.

As depicted in FIG. 2, the measurement hardware is configured to be used with material samples 132a, 132b, 132c, 132d, which may be QC materials, patient test specimens or other specimens as is known in the art. In one embodiment, the material samples are contained in vials which are loaded or inserted into the clinical diagnostic analyzer 110a by an user. The samples may be loaded individually, or in groups, e.g., in a tray that is loaded into the analyzer. In alternative embodiments, the samples may be loaded using an automated loading mechanism, such as a turntable or other mechanism, upon command from the analyzer 110a. Material samples in the form of QC materials are typically provided in lots, with a unique lot number assigned to a lot of samples that are essentially identical as coming from the exact same batch source of material. The analyzer 110a preferably allows information relating to the QC materials to be entered by an user, including statistical information such as a mean or standard deviation for the lot of material. In other embodiments, the information may be obtained over a network or from a server using, for example a QR code on the sample vial or container to uniquely identify the sample or lot.

Input panel/display 130 is in communication with the processor and is operable to present controls to facilitate operation of the analyzer, as well as to present prompts and instructions to an user, and to receive input commands and/or data from the user. The input panel/display 130 is preferably a touch screen having capabilities of displaying text and graphics as well as icons, push buttons, keyboards, and the like to both present data to a user and to receive input from a user of the analyzer. Preferably, the input panel/display 130 includes an audible alert device such as a buzzer or beeper.

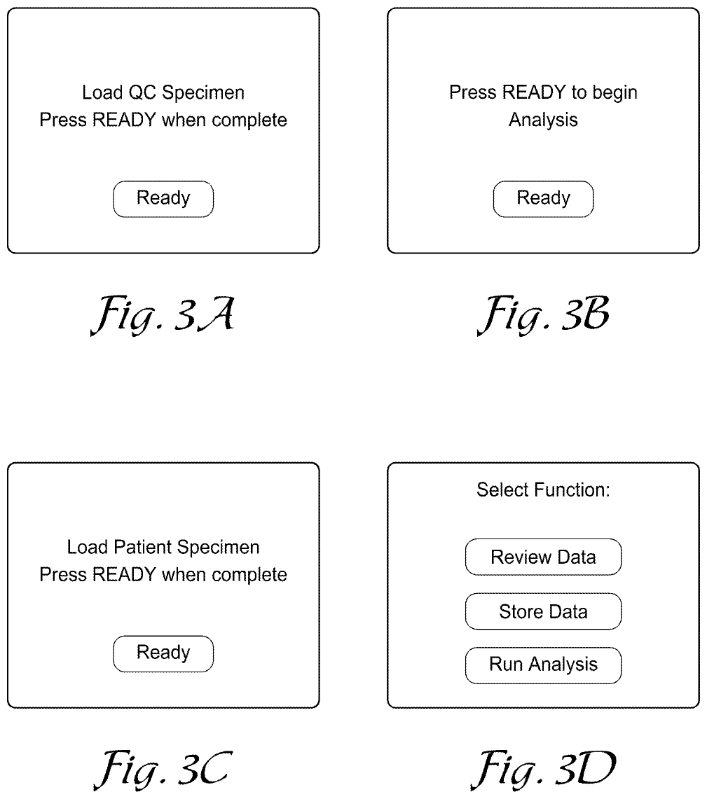

Looking to FIGS. 3A, 3B, 3C, and 3D, for example, the input panel/display may present prompts to a user to load a QC specimen and press a READY button once completed (FIG. 3A), to begin an analysis (FIG. 3B), to load a patient specimen (FIG. 3C) or to select another desired function, such as reviewing data, storing data, or running an analysis (FIG. 3D). It should be understood that the clinical diagnostic analyzer 100a may have multiple programs and functions available, a menu or selection prompt is preferably presented to guide a user through the operation of the analyzer and the selection of desired functions and operations.

Clinical diagnostic analyzer 110a may be any type of analyzer known in the art, such as biochemistry analyzers, hematology analyzers, immune-based analyzers, or any other clinical diagnostic analyzer known in the art. Preferably, analyzer 110a is configured to test quality control materials having known properties to allow users to determine the accuracy of the analyzer and to provide assurance to users that the analyzer is operating within allowable tolerances. Clinical diagnostic analyzer 110a may be configured for use with various quality control materials, whether in liquid or lyophilized form, and may be configured for use in the immunoassay, serum chemistry, immunology, hematology, and other fields.

Looking to FIGS. 1 through 3 in combination, in typical use in performing a test on a patient specimen, the analyzer 110a prompts an user to load a patient specimen as depicted in FIG. 3C, and to perform an analysis as depicted in FIG. 3B once the sample is loaded. Upon completion of the test, the analyzer may prompt the user to store or review the data as seen in FIG. 3D. Similarly, the analyzer may guide an user to test QC material as depicted in FIG. 3A.

It should be understood that the operation of the analyzer 100a may be performed locally, at the analyzer, or that the operation may be coordinated thorough the server 112 when the analyzer is operated in a system 100 as depicted in FIG. 1 It should be further understood that any data may be stored locally on the analyzer 110a, on the server 112 or database 114, and that the data may be made available throughout the system 100 and over the network 116 so that remote servers and analyzers may likewise access the stored data. Similarly, analyses may be run on the analyzer itself, on the server, or may be distributed among multiple analyzers and/or servers.

In embodiments of the invention described herein, the analyses performed on multiple analyzers and the data collected may be analyzed in combination to provide an output or result based on data collected across multiple analyzers.

While known methods of performing crossover studies rely on manual collection and analysis of data over a period of at least twenty days, the system and method of the present invention performs an automated crossover study using either a single clinical diagnostic analyzer, multiple diagnostic analyzers, or single or multiple clinical diagnostic systems to perform crossover studies in as little as one day with greater accuracy than provide by conventional methods.

With the configuration of the clinical diagnostic analyzers and system set forth, systems and methods for conducting automated crossover studies in accordance with the present invention will now be described.

As discussed above, conducting a crossover study involves determining the statistical behavior of a new lot of control material, namely, the mean and standard deviation of the new material. Because the clinical diagnostic analyzers used to analyze test specimens and patient specimens, until a laboratory can ascertain the parameters of the new control material, it cannot be certain of the accuracy of the results of analyses performed on actual specimens.

In order to determine the mean and standard deviation of the new lot of control material, a laboratory must initially define several process parameters relating to the old, or prior, lot of control material used or in current use, and relating to the new lot of control material to be used, as follows:
SD_{old}—is the standard deviation of the old lot of control material.
MEAN_{old}—is the mean of the old lot of control material.
CV_{old}—is the coefficient of variation of the old lot of control material
SD_{new}—is the standard deviation of the new lot of control material
MEAN_{new}—is the mean of the new lot of control material
CV_{new}—is the coefficient of variation of the new lot of control material.

Note that coefficient of variation CV is sometimes referred to as a relative standard deviation (RSD) and can be expressed as the ratio of the standard deviation to the mean.

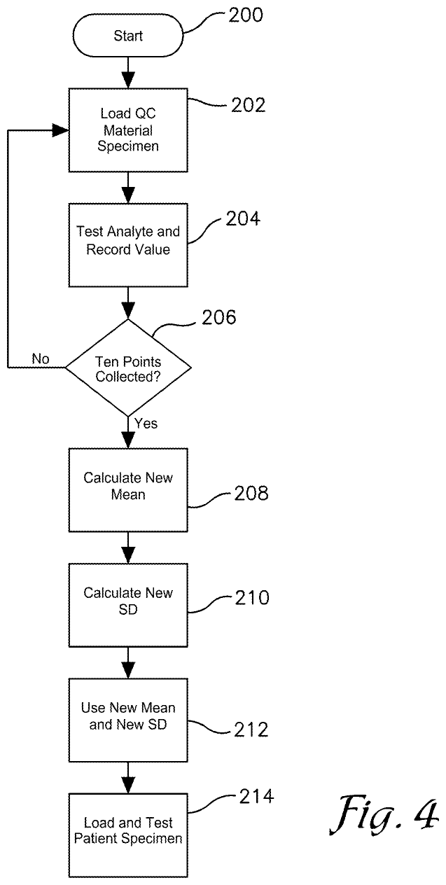

With the initial parameters for conducting an automated crossover study set forth, the general steps for implementing on method of an automate crossover study are depicted in the flow diagram of FIG. 4. Each step will first be described generally; a more detailed description of each step in the process is set forth below.

Looking first to FIG. 4, an automated method for calculating a mean and standard deviation of a new lot of control materials by collecting ten data points over a period of ten days from a single clinical diagnostic analyzer, begins at block 200 where the process starts. A

At block 202, the QC material to be tested is loaded into the analyzer. The material may be loaded by an user in response to a prompt from the analyzer, as depicted in FIG. 3A, or may be automatically loaded in response to a command from the analyzer by an automated loading mechanism.

At block 204, an analyte—i.e., a portion of the new control material, extracted by the measurement hardware in the analyzer—is tested by the clinical diagnostic analyzer and a value is determined.

At block 206, if ten data points have not been collected (i.e., ten days of testing have not been completed), then the steps at blocks 202 and 204 are repeated on the following day, with another test being performed on the analyte. Thus, the steps at block 202 and 204 are repeated until ten data points have been collected, at which time the method proceeds to block 208.

At block 208, the new mean for the new control material (MEAN_{new}) is calculated as MEAN_{new}=(Σ_{i=1 to 10} valueᵢ/10), i.e., the new mean is the sum of the ten collected data point values, or control values, divided by ten.

At block 210, the standard deviation for the new control material (SD_{new}) is calculated as SD_{new}=MEAN_{new}*CV_{old}/100.

At block 212, with the new mean and the new standard deviation calculated, those values are used by the analyzer for subsequent testing, and the analyzer may be used to proceed with testing of patient specimens at 214.

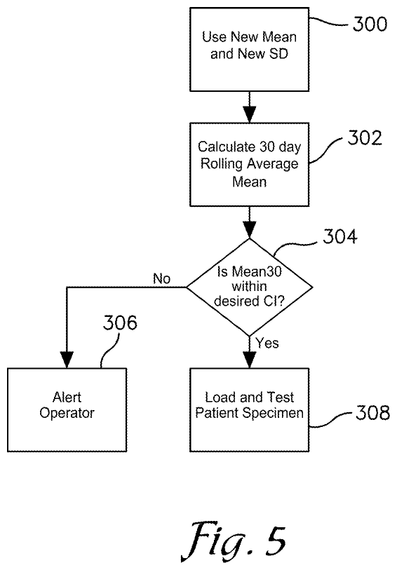

In a further embodiment of the present invention, because of potential inaccuracies in the new mean calculated over the ten-day period as just described, further steps to improve the accuracy of the mean and to detect potential errors are disclosed with reference to FIG. 5.

Additional parameters used in the automated crossover study method of FIG. 5 are:

MEAN₃₀—is the thirty-day rolling average of the MEAN_{new} values. It should be understood that while thirty days is a preferred interval, that other intervals such as twenty days, forty-five days, or ninety days may also be used to achieve a desired filtering or averaging effect.

CI—is the desired confidence interval for evaluating the calculated MEAN₃₀.

Looking to the flow diagram of FIG. 5, at block 300, the new mean and new standard deviation (MEAN_{new} and SD_{new},), calculated as described at block 212 above with respect to FIG. 4, are available for use. It should be understood that in one embodiment, the steps of FIG. 5 are in addition to the steps of FIG. 4, with the steps of FIG. 5 continuing from block 212 of FIG. 4.

At block 302, a thirty-day rolling mean is calculated by taking a thirty-day summation of the control values and dividing by 30, i.e., MEAN₃₀=(Σ_{i=1 to 30} valueᵢ/30).

At block 304, the calculated MEAN₃₀ is checked to determine if it falls within the upper and lower estimates as calculated using the confidence interval CI. Preferably the desired CI is provided by the user, by instructions on the clinical diagnostic analyzer, or obtained over the network from the server.

If the MEAN₃₀ falls outside of the lower or upper limits of the confidence interval CI, then at block 306 an alert to the user is generated, and the clinical diagnostic analyzer stops until corrective action is taken by the user. Preferably, the alert to the user is a message displayed on the input panel/display of the analyzer, in a manner similar to the messages depicted in FIGS. 3A through 3D. In other embodiments, the alert may include an audible alert.

If the MEAN₃₀ does not fall outside of the confidence interval CI, then at block at block 308 a patient specimen is loaded and tested in a manner similar to that previously described.

As can be seen, the systems and methods of the present invention provide an improvement over the generally accepted twenty-day crossover study by an automated method for performing a ten-day crossover study and by improvements to that automated crossover study method.

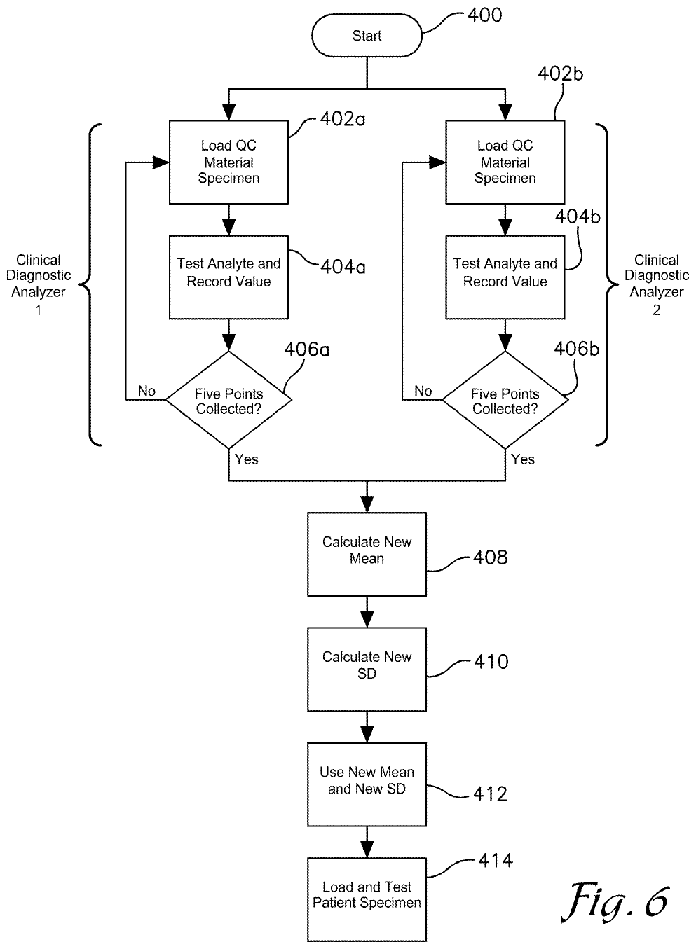

As described above, the twenty-day and ten-day crossover studies require that a single clinical diagnostic analyzer be use to complete the crossover to a new lot of quality control material by testing the QC material once per day, and accumulating the results over the required ten or twenty-day period in the manner described previously. In further embodiments and aspects of the present invention, the ten-day time for conducting an automated crossover study may be greatly reduced as will now be described with respect to FIG. 6.

In accordance with the present invention, using multiple clinical diagnostic analyzers to test the same analyte can mitigate the risk of bias introduced by any given machine, and can reduce the amount of time required to conduct a crossover study. Using multiple machines, running the same analyte, using the same QC mean, the number of test days required may be reduced by a factor inversely proportional to the number of clinical diagnostic analyzers being used. Thus for example, a ten data point crossover study may alternatively be completed by using two machines, each testing the same analyte, over a period of five days, as depicted in the flow diagram of FIG. 6.

Looking to FIG. 6, using two machines, a method similar to that described with respect to FIG. 4 uses the same parameters as described above, namely, SDold, MEANold, CVold, SDnew, MEANnew, and CVnew.

An automated method for calculating a mean and standard deviation of a new lot of control materials by collecting ten data points—five data points each from two separate clinical diagnostic analyzers (clinical diagnostic analyzer 1 and clinical diagnostic analyzer 2 in the figure)—over a period of five days, begins at block 400 where the process starts.

At blocks 402a and 402b, the QC material to be tested is loaded into the respective analyzers. The material may be loaded by an user in response to a prompt from the analyzer, as depicted in FIG. 3A, or may be automatically loaded in response to a command from the analyzer by an automated loading mechanism.

At blocks 404a and 404b, the analyte—i.e., a portion of the new control material, extracted by the measurement hardware in the respective analyzer—is tested by the respective clinical diagnostic analyzer and a value is determined.

At blocks 406a and 406b if five data points have not been collected (i.e., five days of testing have not been completed), then the steps at blocks 402a, 402b and 404a, 404b are repeated on the following day, with another test being performed on the analyte. Thus, the steps at blocks 402a, 402b and 404a, 404b are repeated until five data points have been collected, at which time the method proceeds to block 408.

At block 408, ten data points have been collected—five from each of clinical diagnostic analyzer 1 and clinical diagnostic analyzer 2—and the new mean for the new control material (MEANnew) is calculated as before (with ten total data points having been collected) MEANnew=(Σi=1 to 10 valueᵢ/10), i.e., the new mean is the sum of the ten collected data point values, divided by ten.

At block 410, the standard deviation for the new control material (SDnew) is calculated as SDnew=MEANnew*CVold/100.

At block 412, with the new mean and the new standard deviation calculated, those values are used by the analyzer for subsequent testing, and the analyzer may be used to proceed with testing of patient specimens at 414.

Thus, by employing two clinical diagnostic analyzers to test the same specimen on the same day, the total time to complete the crossover study as compared to the ten day method described with respect to the method described with respect to FIG. 4 has been reduced in half.

It should be apparent to those skilled in the art that the invention as described with respect to the embodiment of FIG. 6 can be extended to further reduce the time required to complete a crossover study. For example, using ten separate clinical diagnostic analyzers, each running a test on the same specimen, a crossover study may be completed in a single day, with each analyzer collecting one data point, and the collective ten data points being used to calculate the mean and standard deviation for the new QC material.

In most cases where the number of clinical diagnostic analyzers used is not an even factor of ten, then the testing load should be adjusted so that the same number of points are collected from each clinical diagnostic analyzer, even it that total number exceeds ten. For example, if three clinical diagnostic analyzers are used in laboratory and the patient specimen testing load for the laboratory is approximately evenly distributed across those three analyzers, then four data points should be collected from each analyzer, for a total of twelve data points. In such cases, the new mean should be calculated as a summation of all twelve of those points, divided by twelve. Balancing or equalizing the number of points collected, rather than truncating to ten points, ensures that bias from a single clinical diagnostic analyzer is not amplified by being effectively weighted more highly in the calculation of the mean.

In other cases, if the QC testing load on a group of clinical diagnostic analyzers is not approximately evenly distributed, then the number of data points to be collected by each analyzer should be weighted to reflect that unequal distribution. For example, if two clinical diagnostic analyzers are used in a laboratory, and the first clinical diagnostic analyzer processes approximately sixty percent of the test samples, and the second clinical diagnostic analyzer processes approximately forty percent of the test samples, then it may be desirable to weight the number of points collected from each analyzer, with the first analyzer providing six data points, and the second analyzer providing four data points. One skilled in the art will appreciate that the method depicted in FIG. 6 may be adjusted accordingly to accommodate that weighted distribution.

While the present invention has been described and illustrated hereinabove with reference to various exemplary embodiments, it should be understood that various modifications could be made to these embodiments without departing from the scope of the invention. Therefore, the invention is not to be limited to the exemplary embodiments described and illustrated hereinabove, except insofar as such limitations are included in the following claims.

What is claimed and desired to be secured by Letters Patent is as follows:

1. A clinical diagnostic analyzer for conducting automated crossover studies, comprising:
a processor;
measurement hardware in communication with the processor and configured to measure properties of a specimen;
a memory device having stored thereon executable instructions that, when executed by the processor, cause the clinical diagnostic analyzer to perform operations comprising:
loading a specimen from a new lot of quality control material into the measurement hardware;
analyzing the specimen at periodic intervals to obtain a data value corresponding to an attribute of the specimen;
obtaining and storing at least ten consecutively obtained data values corresponding to analyses performed at consecutive periodic intervals;
calculating a mean for the new lot of quality control material based on the stored data values;
calculating a standard deviation based on the calculated mean and a coefficient of variation of an old lot of quality control material;
storing the calculated mean and the calculated standard deviation in the memory device for use in subsequent analyses;
loading and testing an analyte from a patient specimen using the stored mean and standard deviation.

2. The clinical diagnostic analyzer of claim 1, wherein the memory device includes instructions, that when executed, further cause the clinical diagnostic analyzer to perform operations comprising:
calculating a thirty-day rolling average of the calculated mean; and
storing the thirty-day rolling average of the calculated mean in the memory device for use in subsequent analyses.

3. The clinical diagnostic analyzer of claim 2, wherein the memory device includes instructions, that when executed, further cause the clinical diagnostic analyzer to perform operations comprising:
comparing the calculated thirty-day rolling average to a predetermined confidence interval; and
alerting a user if the calculated thirty-day exceeds an allowable variation based on the confidence interval.

4. The clinical diagnostic analyzer of claim 1, further comprising an input panel and display operable to present information and data from the processor to a user and to accept input and selections from a user.

5. The clinical diagnostic analyzer of claim 4, wherein the memory device includes instructions, that when executed, further cause the clinical diagnostic analyzer to perform operations comprising:
presenting a prompt on the input panel and display to a user to load an analyte into the measurement hardware; and
accept an input from the user indicating that the analyte has been loaded.

6. A system for conducting automated crossover studies, comprising:
a server comprising a processor, a memory and a database;
a plurality of clinical diagnostic analyzers in communication with the server, wherein each of the plurality of clinical diagnostic analyzers comprises:
a processor;
measurement hardware in communication with the processor and configured to measure properties of a specimen;
a memory device having stored thereon executable instructions that, when executed by the processor, cause the clinical diagnostic analyzer to perform operations comprising:
loading a specimen from a new lot of quality control material into the measurement hardware;
wherein the memory of the server has stored thereon executable instructions that, when executed by the server processor, cause the server to perform operations comprising
receiving, from the plurality of clinical diagnostic analyzers, the stored obtained values;
calculating a mean for the new lot of quality control material based on the received values;
calculating a standard deviation based on the calculated mean and a coefficient of variation of an old lot of quality control material;
storing the calculated mean and the calculated standard deviation in the memory for use in subsequent analyses;
prompting a user of one or more of the plurality of clinical diagnostic analyzers to load and test an analyte from a patient specimen using the stored mean and standard deviation.

7. The system of claim 6, wherein the server memory includes instructions, that when executed, further cause the server to perform operations comprising:
calculating a thirty-day rolling average of the calculated mean; and
storing the thirty-day rolling average of the calculated mean in the memory, the database, or combinations thereof, for use in subsequent analyses.

8. The system of claim 7, wherein the server memory includes instructions, that when executed, further cause the server to perform operations comprising:
comparing the calculated thirty-day rolling average to a predetermined confidence interval; and
alerting a user if the calculated thirty-day exceeds an allowable variation based on the confidence interval.

9. The system of claim 6, wherein each of the plurality of clinical diagnostic analyzers comprises an input panel and display, and wherein the server memory includes instructions, that when executed, further cause the server to perform operations comprising:
transmitting an instruction to at least one of the plurality of clinical diagnostic analyzers to present a prompt on the input panel and display to a user to load an analyte into the measurement hardware; and
accept an input from the user of the at least one of the plurality of clinical diagnostic analyzers indicating that the analyte has been loaded.

10. A method for conducting automated crossover studies, comprising:
loading a specimen from a new lot of quality control material into measurement hardware of a clinical diagnostic analyzer;
analyzing the specimen at periodic intervals to obtain a data value corresponding to an attribute of the specimen;
obtaining and storing at least ten consecutively obtained data values corresponding to analyses performed at consecutive periodic intervals;
calculating a mean for the new lot of quality control material based on the stored data values;
calculating a standard deviation based on the calculated mean and a coefficient of variation of an old lot of quality control material;
storing the calculated mean and the calculated standard deviation for use in subsequent analyses;
loading and testing an analyte from a patient specimen using the stored mean and standard deviation.

11. The method of claim 10, further comprising:
calculating a thirty-day rolling average of the calculated mean; and
storing the thirty-day rolling average of the calculated mean in the memory device for use in subsequent analyses.

12. The method of claim 11, further comprising:
comparing the calculated thirty-day rolling average to a predetermined confidence interval; and
alerting a user if the calculated thirty-day exceeds an allowable variation based on the confidence interval.

13. The method of claim 10, wherein the clinical diagnostic analyzer comprises a plurality of clinical diagnostic analyzers in communication over a network.

14. The method of claim 13, wherein obtaining and storing at least ten consecutively obtained data values corresponding to analyses performed at consecutive periodic intervals comprises obtaining and storing obtained data values from each of the plurality of clinical diagnostic analyzers.

15. The method of claim 10, further comprising:
prompting, on the clinical diagnostic analyzer, a user to load a specimen, and
receiving input from a user confirming the specimen has been loaded.
